(19) United States
(12) Patent Application Publication
(10) Pub. No.: US 2005/0064402 A1
(43) Pub. Date: Mar. 24, 2005

(54) Tumor marker and methods of use

(76) Inventors: Susan McDonald Goldsworthy, Durham, NC (US); David Erik Jensen, Durham, NC (US); Traci Ann Mansfield, New Haven, CT (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/398,058

(22) PCT Filed: Oct. 1, 2001
(86) PCT No.: PCT/US01/30682

Related U.S. Application Data
(60) Provisional application No. 60/237,605, filed on Oct. 3, 2000.

(51) Int. Cl. C12Q 1/68

(52) U.S. Cl. 435/6

(57) ABSTRACT

The use of the protein kinase “PBK” (PDZ-binding kinase) as a tumor marker in various tissue types is discussed. Methods of detecting overexpression of the protein, and kits useful therefore, are described.

### FIELD OF THE INVENTION

The present invention relates to proteins that can be utilized as tumor markers for human cancers, and to methods of using the same.

### BACKGROUND OF THE INVENTION

Various biochemical and immunological entities are useful as markers to indicate the presence, nature or extent of cancer in mammals. Marker molecules produced by cancers include monoclonal immunoglobulins, hormones, secreted serum proteins, antigens, enzymes and isoenzymes, cell surface markers, glycoproteins and carbohydrates, and extracellular matrix proteins and mucins. Cancer markers may be categorized as soluble markers, which appear in bodily fluids such as blood, plasma, serum, effusions, and urine. Other cancer markers are cell-associated proteins and nucleic acids that characteristically are not released into serum or other body fluids in any significant amounts. Cell- or tissue-associated tumor markers are detectable in tissue samples containing cancerous cells, or in biological samples that contain such cells (e.g., sputum samples containing shed bronchial epithelial cells).

A cancer marker may be an oncodevelopmental marker that reflects the less differentiated state of the cancer cell (compared to non-cancerous or normal cells), or it may reflect molecular changes in the cell due to the activation of cell division pathways or inhibition of cell death pathways. The expression of these markers in cells may be utilized to identify cells as abnormal or as cancerous (having malignant potential).

In cancer, prognosis and choice of treatment varies depending on the extent of a cancerous growth as well as its histological type. The clinical diagnosis of cancer rarely depends on a single test; multiple markers may be assessed to aid in treatment planning. There continues to be a need to identify novel tumor markers to aid in the detection, prognosis and monitoring of human cancers.

### SUMMARY OF THE INVENTION

An aspect of the present invention is a method of detecting mRNA encoding a marker protein associated with a cancer that is characterized by over-expression of PDZ-binding kinase (PBK) compared to non-cancerous cells. A test sample of tissue is obtained, and mRNA from the test sample is contacted with a nucleotide probe that specifically hybridizes to mRNA encoding PBK, to form a hybridization product. The amount of hybridization product is measured and compared to that which would be expected in a sample of non-malignant tissue.

A further aspect of the present invention is a method of detecting mRNA encoding a marker protein associated with a cancer that is characterized by over-expression of PDZ-binding kinase (PBK) compared to non-cancerous cells, where mRNA is obtained from a test sample of tissue and amplified using a nucleic acid sequence primer that specifically binds to mRNA encoding PBK to form an amplified product. The amount of amplified product is measured and compared to that in non-malignant cells. A finding that the amount of amplified product produced by the test sample is greater than that produced by non-malignant control cells indicates the presence of cancerous cells in the test sample.

A further aspect of the present invention is a method of screening a sample of prostate tissue for malignancy, by measuring the amount of PBK mRNA in the sample tissue. An elevated level of PBK mRNA, compared to non-malignant prostate tissue, indicates that the sample tissue is malignant.

A further aspect of the present invention is a method of screening a sample of prostate tissue for malignancy, by measuring the amount of PBK protein in the sample tissue. An elevated level of PBK in the sample, compared to non-malignant prostate tissue, indicates that the sample tissue is malignant.

A further aspect of the present invention is a method of screening a sample of breast tissue for malignancy, by measuring the amount of P13K mRNA in the sample tissue. An elevated level of PBK mRNA in the sample, compared to non-malignant breast tissue, indicates that the sample tissue is malignant.

A further aspect of the present invention is a method of screening a sample of breast tissue for malignancy, by measuring the amount of PBK protein in the sample tissue. An elevated level of PBK in the sample, compared to non-malignant breast tissue, indicates that the sample tissue is malignant.

A further aspect of the present invention is a method of screening a test compound for the ability to inhibit PBK activity in cells expressing PBK. The method comprises exposing cells to a test compound, detecting the amount of PBK activity present in the cells; and comparing the amount of PBK activity in the exposed test cells to that in control cells that were not exposed to the test compound. A decreased amount of PBK activity in the exposed test cells compared to control cells indicates that the test compound inhibits PBK activity in malignant cells.

A further aspect of the present invention is a kit for detecting mRNA encoding a marker protein associated with a human cancer characterized by over-expression of PDZ-binding kinase (PBK) compared to non-cancerous cells. The kit includes a labeled nucleic acid molecule at least 20 nucleotides in length, complementary to a human PBK mRNA sequence, and able to specifically hybridize to the human PBK mRNA sequence to form a labeled hybridization product. The kit further includes printed instructions setting forth levels of PBK mRNA expected in cancerous cells of at least one human tissue type.

A further aspect of the present invention is a kit for detecting a marker protein associated with a human cancer characterized by over-expression of PDZ-binding kinase (PBK) compared to non-cancerous cells. The kit includes a labeled antibody that specifically binds to human PBK, and printed instructions setting forth levels of PBK expected in cancerous cells of at least one human tissue type.

A further aspect of the present invention is a transgenic or mutagenized mouse whose genome comprises a disruption in at least one allele of its endogenous PDZ-binding kinase (PBK) gene. The disruption prevents the expression of a functional PBK protein, and results in the transgenic or mutant mouse exhibiting decreased levels of PBK activity (as compared to a wild-type mouse).

A further aspect of the present invention is a method for producing a transgenic mouse exhibiting decreased levels of PBK activity relative to a wild-type mouse. The method comprises introducing a PBK targeting vector into a mouse embryonic stem cell; introducing the mouse embryonic stem cell into a mouse blastocyst; and transplanting the mouse blastocyst into a pseudopregnant mouse.

A further aspect of the present invention is a transgenic mouse whose genome comprises a transgene comprising a DNA sequence encoding all or part of human PBK, with the DNA sequence operably linked to a tissue-specific promoter.

### BRIEF DESCRIPTION OF THE DRAWINGS

FIG. 1 graphs the expression of PBK mRNA in normal human tissues. Relative abundance of P13K mRNA is assessed across the various tissues and is a unitless measure of the level of PBK mRNA in the sample.

FIG. 2 graphs the expression of PBK mRNA in human lung tumor samples. Ten-fold overexpression or greater (compared to expression levels of PBK mRNA in a paired, non-cancerous lung tissue sample) was found in thirteen (57%) of the twenty-three lung tumor samples examined.

FIG. 3 graphs the expression of PBK mRNA in human prostate tumor samples, compared to paired normal prostate samples. Overexpression of 10× or greater was found in seven (78%) of nine prostate tumor samples examined.

FIG. 4 graphs the expression of PBK mRNA in human colon tumor samples, compared to paired normal colon tissue samples. Overexpression of 10× or greater was found in five (25%) of twenty colon tumor samples examined.

FIG. 5 graphs the expression of PBK mRNA in human breast tumor samples, compared to paired normal breast tissue samples. Overexpression of 10× or greater was found in six (67%) of nine breast tumor samples examined.

### DETAILED DESCRIPTION

A recently identified protein kinase has been identified by the present inventors as useful as a tumor marker. The kinase was termed “PBK” (PDZ-binding kinase) by Gaudet et al., Proc. Natl. Acad. Sci. USA 97(10):5167 (2000), and alternatively was called “TOPK” (T-LAK cell-originated protein kinase) by Abe et al., J. Biological Clem. 275(28):21525 (2000). The kinase is referred to as PBK TDZ-binding kinase) herein. PDZ domains mediate protein-protein interactions at specialized subcellular sites, such as epithelial cell tight junctions and neuronal post-synaptic densities. Most PDZ domains bind the extreme carboxyl-terminal sequences of their protein partners. Fuh et al., J Biol Chem 275(28):21486 (2000).

PBK is a 322-amino acid serine/threonine kinase having the following amino acid sequence:

| | | |
|---|---|---|
| MEGISNFKTP SKLSEKKKSV LCSTPTINIP ASPFMQKLGF GTGVNVYLMK RSPRGLSHSP | 60 | (SEQ ID NO:2) |
| WAVKKINPIC NDHYRSVYQK RLMDEAKILK SLHHPNIVGY RAFTEANDGS LCLAMEYGGE | 120 | |
| KSLNDLIEER YKASQDPFPA AIILKVALNM ARGLKYLHQE KKLLHGDIKS SNVVIKGDFE | 180 | |
| TIKICDVGVS LPLDENMTVT DPEACYIGTE PWKPKEAVEE NGVITDKADI FAFGLTLWEM | 240 | |
| MTLSIPHINL SNDDDDEDKT FDESDFDDEA YYAALGTRPP INMEELDESY QKVIELFSVC | 300 | |
| TNEDPKDRPS AAHIVEALET DV | | |

The amino acid sequence reported by Gaudet et al. Proc. Natl. Acad. Sci. USA 97(10):5167 contains a serine (S) at position 107 (GenBank Accession No. AF189722; position 107 indicated by bold underlined type in the sequence above). Both Abe et al., J. Biological Chem. 275(28):21525 and the present inventors identified an asparagine (N) at this position (SEQ ID NO:3). PBK has characteristic protein kinase subdomains and a C-terminal PDZ-binding T/SXV motif; the consensus D-X-K-X-X-N sequence for serine/threonine or dual specificity kinases is found at residues 167-172 of SEQ ID NO:2 (underlined, above). PBK binds specifically to PDZ2 of the human hDlg (the human homologue of Drosophila Discs-Large (Dlg) tumor suppressor protein). Gaudet et al. A full-length cDNA encoding the PDZ kinase is provided herein as SEQ ID NO:1.

Tissue distribution of PBK kinase mRNA expression was reported by Abe et al. as specific for testis, T-LAK (lymphokine-activated killer T) cells, activated lymphoid cells and RPMI 1788 B-cell lymphoma cells. Gaudet et al. state that mRNA for PBK was most abundant in placenta, and was absent from adult brain tissue.

The terms “cancer”, “malignancy” and “malignant tumor” are used to refer to any of various types of malignant neoplasms, as will be understood by one of ordinary skill in the art. Neoplasm refers to an abnormal growth of tissue produced by cellular proliferation. Neoplasms typically show a lack of structural organization and/or functional coordination with normal tissue, and may form a distinct mass of tissue. Neoplasms may be either benign or malignant. “Malignant” refers to the ability of a neoplasm to invade surrounding tissues. Malignant growths are usually capable of metastasizing to sites that are anatomically distant from the primary site.

As used herein, a malignant tumor cell or cancer cell refers to a cell that has undergone a malignant transformation event. Malignant transformation refers to a change in a cell that greatly increases the cell's ability to produce cancerous growths; malignant transformation may be identified by changes in the cell's growth characteristics, histology, and/or the morphology of the cell, as is known in the art.

The term “solid tumor” is used in its medically accepted sense, and does not include cancers of the blood such as leulemias.

As used herein, a “cancer marker” or “tumor marker” is an indicator of the presence of, or extent of, a cancerous growth or tumor. It is known in the art that a tumor marker need not be either 100% specific nor 100% selective to be useful in clinical practice. Clinical cancer diagnosis rarely depends on the assessment of a single marker; additional diagnostic, laboratory and clinical procedures and observations typically are considered when making a diagnosis of human cancer.

The term ‘cancer diagnosis’ refers to the identification of the presence of a cancer or malignant tumor, as well as the differentiation of benign from malignant disease. As used herein, prognosis refers to the assessment or prediction of how well or how poorly a patient will fare in terms of response to therapy, relapse, survival time, or other outcome measures as are known in the art. As used herein, monitoring refers to the repeated assessment of a patient to detect signs of disease progression; monitoring may be carried out during treatment to assess the effects of the treatment, or may be carried out after treatment to detect signs of minimal residual disease, renewed disease, or continued disease progression.

Prostate cancer refers to any histological type of cancer arising from a prostate cell, including metastatic tumors distant from the prostate but originating from a prostate cancer. Most prostate cancers are adenocarcinomas, although carcinosarcomas, transitional cell, small cell, primary sarcomas, and other histological types occur less frequently. Prostate cancer is a well-recognized disease entity.

Breast cancer refers to any histological type of cancer arising from a breast tissue cell, including metastatic tumors distant from the breast but originating from a breast cancer. Most breast cancers arise from epithelial cells; other histologic types of breast cancers have been described. Breast cancer is a well-recognized disease entity.

Tumor markers may be assayed within the tissue suspected of being diseased (i.e., of containing malignant or cancerous cells), or by indirect collection of exfoliated cellular material from various sources such as sputum, urine, tumor fluid, or nipple fluid.

The present inventors have determined that PBK mRNA is overexpressed in tumor tissue samples, compared to paired normal tissue samples. Accordingly, detection and/or quantitative measurement of PBK or nucleic acid molecules encoding PBK is useful in detecting malignancies. In one embodiment of the invention, the present methods are used to detect the presence of prostate cancer cells in prostate tissue samples. In another embodiment of the present invention, the methods are used to detect the presence of breast cancer cells in breast tissue samples. In still further embodiments of the present invention, the methods are used to detect colon cancer cells in colon tissue samples, and to detect lung cancer cells in lung tissue samples.

The present invention includes the use of PBK protein and its allelic variants, and nucleic acid molecules encoding PBK (and variants thereof) as markers to detect cancerous tissue. These markers are characteristically present in increased levels in cancer cells, compared to levels in normal cells of the same tissue type. In non-cancerous tissue or non-proliferative tissue, PBK markers may be present in low or undetectable levels. PBK “markers” include the PBK protein and variants and fragments thereof, and nucleic acid molecules encoding PBK or fragments or variants thereof. As used herein, an “increased level” of a PBK marker refers to a level that is increased over a predetermined standard, or increased over the level in a control sample. The pre-determined standard may be based on the typical measurement of a PBK marker in healthy (non-cancerous) tissue samples of the same type as that being tested for a suspected malignancy. The pre-determined standard may be zero or undetectable, or it may be a positive amount. Alternatively, in testing a subject, the pre-determined standard may be based on an earlier test result from that subject, or the standard may be based on measurement of PBK in normal tissue from that subject.

Accordingly, a method of the present invention comprises isolating cells, tissues or extracts thereof from a human and determining the comparative level of a PBK marker, as an indicator of diseases associated with increased PBK activity, such as cancer, proliferative disorders, pathologic angiogenesis, and inflammation. A preferred method of the present invention comprises isolating cells, tissues or extracts thereof from a human and determining the comparative level of a PBK marker, which is an aid in predicting the malignant status of the cells or tissues.

It is well known in medical diagnostics that a test result may vary somewhat from a normal standard even where no disease is present. Thus a normal standard may include a range of values. The level of a marker that is accepted as indicating disease (a “diagnostic level”) may be several times the normal level. Known statistical methods may be employed to determine a diagnostic level of a PBK marker in a particular tissue or for a particular disease, and a diagnostic level which suggests the presence of disease with some certainty. For example, the diagnostic level of PBK mRNA may be set at one and a half times normal levels for the target tissue (1.5×), twice normal levels (2×), five times normal levels (5×), ten times normal levels (10×), one hundred times normal levels (100×), five hundred times normal levels (500×), one thousand times normal levels (1,000×), one thousand five hundred times normal levels (1,500×), or more. Typically the diagnostic level is set at a point where it is statistically more likely than not that a subject has the target disease when the diagnostic level of disease marker is found.

The level of PBK marker that is considered as an indicator of disease may vary among the target tissues tested. Normal PBK marker standards, and PBK marker diagnostic levels can be determined for a specific tissue type or disease using routine diagnostic and clinical testing methods as are known in the art. Any number of protocols may be used to obtain data to establish a standard or diagnostic level for the methods of the present invention, as will be apparent to those skilled in the art.

The methods of the present invention may be employed with subjects suspected of having a disease state that has been associated with increased PBK markers, including but not limited to: solid tumors, prostate cancer, lung cancer, colon cancer, and breast cancer. The present methods may be used to monitor subjects who have been previously diagnosed with the target condition, to monitor subjects undergoing treatment for the target condition, or to screen subjects who have not been previously diagnosed with the target condition. The methods disclosed herein are particularly suited to screening prostate tissue samples and breast tissue samples to determine if the cells therein are cancerous.

As used herein, methods of screening and diagnosis does not indicate that such methods are 100% specific or 100% sensitive in detecting the target disease. In testing multiple samples, however, a majority of the samples testing at or above the diagnostic level will contain malignant tissue. The specificity and/or sensitivity of the present methods may vary depending on the condition being tested for, the biological sample being used, the general health of the subject, and other factors, as will be apparent to those skilled in the art. It is also readily apparent that the relative number of malignant and non-malignant cells in a tissue sample will affect the test results; samples that contain small foci of malignant cells within a larger sample of non-malignant cells are more likely to test false-negative than samples containing essentially all malignant cells.

In one embodiment of the present invention, the amount of PBK mRNA present in a tissue sample suspected of containing cancerous cells is measured and compared to a predetermined standard, or compared to the amount of PBK mRNA present in a sample of normal (non-cancerous) tissue of the same type. Various methods of measuring or comparing mRNA levels are known in the art. A preferred method for quantitative measurement of mRNA utilizes reverse transcriptase polymerase chain reaction (RT-PCR) techniques as are known in the art; a particularly preferred method utilizes TAQMAN® (PE Applied Biosystems, Foster City, Calif.), a system of real-time fluorescent-probe polymerase chain reaction (PCR). This method uses a fluorogenic probe complementary to the target sequence, which is added to the PCR reaction mixture. The probe comprises an oligonucleotide with a reporter and quencher dye attached. If the target nucleic acid is present during PCR, the probe anneals specifically between the forward and reverse primer sites. The polymerase cleaves the probe, causing an increase in the fluorescent intensity of the reporter dye. Fluorescent emission is then recorded and/or measured. See, e.g., Orlando, C., P., Pinzani, and M., Pazzagli. “Developments in quantitative PCR.” Clin Chem Lab Med 36 (1998): 255-269; Brink et al., “Comparative quantification of IL-1beta, IL-10, IL-10r, TNFalpha and IL-7 mRNA levels in UV-irradiated human skin in vivo”, Inflamm Res 49(6):290 (2000). Optimization of the PCR reaction is required for each primer and probe set, as is known to those skilled in the art. Commercial software is available to assist in selecting primers and probes (e.g., Perkin Elmer PE/ABD Primer Express software). See, e.g, Heid et al., Real-time quantitative PCR. Genome Res 6:986 (1996); Livak et al., PCR Methods Appl 4:357 (1995); Holland et al., Proc Natl Acad Sci USA 88:7276 (1991); Gelmini et al., Clin. Chem43:752 (1997); Marcucci et al., Leukemia 12 (1998).

Polymerase chain reaction (PCR), as is known in the art, allows measurement of DNA or mRNA in tissue samples, including fixed tissue-samples, sloughed cells, and bone marrow aspirates. Many variations of PCR technique are known; one skilled in the art will be able to determine suitable PCR conditions based upon the nature of the DNA or mRNA being analyzed, the tissue, and the desired sensitivity of the assay.

In a further embodiment of the invention, the amount of PBK protein present in a tissue sample suspected of containing cancerous cells is measured and compared to a predetermined standard, or compared to the amount of PBK protein present in a sample of normal (non-cancerous) tissue of the same type. Methods of quantitative detection of proteins are known in the art, including but not limited to use of labeled monoclonal or polyclonal antibodies that specifically bind to the target protein. The amount of PBK protein may be determined as an amount of PBK protein per volume of sample, or as a percentage of total protein.

Antibodies useful in the present invention include those which bind specifically to a peptide of SEQ ID NO:1, SEQ ID NO:2 or SEQ ID NO:3, and fragments of such antibodies, which fragments bind specifically to a peptide of SEQ ID NO:1, SEQ ID NO:2 or SEQ ID NO:3. Such antibodies and antibody fragments may be produced by a variety of techniques as are known in the art. The term “antibodies” as used herein refers to all types of immunoglobulins, including IgG, IgM, IgA, IgD, and IgE. Of these, IgM and IgG are preferred. The antibodies may be monoclonal or polyclonal, and may be of any species of origin, including but not limited to human, mouse, rat, rabbit and horse, and may be chimeric antibodies.

Monoclonal antibodies used in the present methods may be produced in a hybridoma cell line according to the techniques of Kohler and Milstein, Nature, 265:495 (1975) and other techniques as are known in the art. Monoclonal antibodies specific for a tumor marker allow the use of immunoradiometric, immunoenzymatic, or immunohistochemical techniques (as are known in the art) to detect that tumor marker.

Antibodies to cancer markers are useful to detect the presence of the marker in histologic sections, to distinguish tumor cells from normal cells. Immunohistologic staining also allows more precise definition of lesions, e.g., to assess whether a surgically excised sample has diseased tissue at the excision margins. Thus, PBK antibodies are particularly useful when they are suitable for use in paraffin-embedded or formalin-fixed tissues.

Antibodies useful in the present methods may be labeled to produce a detectable signal, as is known in the art. Such labels include radioactive labels, fluorescent labels and enzyme labels. Examples of suitable immunoassays include radioimmunoassays, immunofluorescent assays, enzyme-linked immunoassays, and the like. Antibodies described herein may further be conjugated to a solid support suitable for use in a diagnostic assay (e.g., beads, plates, slides or wells formed from materials such as latex or polystyrene).

Samples taken from subjects for use in the methods disclosed herein are generally samples of tissues, or samples of biological fluids that contain cells from the tissue to be tested. Samples may be obtained by any method as is known in the art, including but not limited to surgical excision, needle aspiration biopsy, and punch biopsy. Samples may undergo additional conventional preparation steps prior to detection of PBK protein or nucleic acid molecules, as are known in the art. Such steps may include, for example, the addition of preservatives or the fixing of a tissue sample in a matrix.

The methods disclosed herein provide an assessment of the amount of PBK present in a biological sample, either by measurement of the PBK protein, a fragment or variant of that protein, or a nucleic acid entity encoding the PBK protein (or a fragment or variant thereof). In a preferred embodiment, the amount of PBK mRNA is measured. However, any suitable method of detecting the comparative amounts of a PBK marker in malignant and normal (non-malignant) cells may be used in the present methods, as would be apparent to one skilled in the art.

The present invention also provides for use of variants of PBK proteins or peptides as disclosed herein. Natural variants of the PBK protein sequences provided herein may differ by conservative amino acid sequence differences or by minor non-conservative sequence differences. Variant PBK proteins or peptides preferably have at least 70%, 75%, 80%, 85%, 90%, 95%, 97%, 98% or greater sequence identity to the PBK sequences provided herein (SEQ ID NOs: 2 and 3). Conservative amino acid substitutions include substitutions within the following groups:

Glycine, alanine;
Valine, isoleucine, leucine;
Aspartic acid, glutamic acid;
Asparagine, glutamine;
Serine, threonine;
Lysine, arginine;
Phenylalanine, tyrosine

The phrases “percent identity” or “percent homology” refer to the percentage of sequence similarity found in a comparison of two or more amino acid or nucleic acid sequences. Percent identity can be determined electronically, e.g., by using the MegAlign™ program (DNASTAR, Inc., Madison Wis.). The MegAlign™ program can create alignments between two or more sequences according to different methods, e.g., the clustal method. (See, e.g., Higgins, D. G. and P. M. Sharp (1988) Gene 73: 237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between nucleic acid sequences can also be counted or calculated by other methods known in the art, e.g., the Jotun Hein method. (See, e.g., Hein, J. (1990) Methods Enzymol. 183: 626-645.)

The term “substantial sequence similarity” as used herein means that DNA, RNA or amino acid sequences which have slight and non-consequential sequence variations from those disclosed herein are considered to be equivalent. Molecules with substantial sequence similarity will be functionally equivalent, i.e., function in substantially the same manner to produce substantially the same compositions and results as the sequences specifically disclosed herein. Molecules with substantial sequence similarity typically have at least about 70% sequence identity, and preferably are at least 75%, 80%, 85%, 90%, 95%, or even 97% or more similar in sequence.

Use of the phrase “isolated” in reference to nucleic acid, peptide, or protein molecules means that the molecules have been separated from their natural in vivo cellular components through the efforts of human beings.

As used herein, the term “stringent conditions” refers to conditions which permit hybridization between polynucleotides and nucleotide molecules that encode PBK proteins as disclosed herein. Stringent conditions can be defined by salt concentration, the concentration of organic solvent (e.g., formamide), temperature, and other conditions well known in the art. In particular, stringency can be increased by reducing the concentration of salt, increasing the concentration of formamide, or raising the hybridization temperature.

For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30 degrees C., more preferably of at least about 37 degrees C., and most preferably of at least about 42 degrees C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30 degrees C. in 750 mM NaCl, 75 mM trisodium citrate, and 1% SDS. In a more preferred embodiment, hybridization will occur at 37 degrees C. in 500 mM NaCl, 50 mM trisodium citrate, 1% SDS, 35% formamide, and 100 μg/ml denatured salmon sperm DNA (ssDNA). In a most preferred embodiment, hybridization will occur at 42 degrees C. in 250 mM NaCl, 25 mM trisodium citrate, 1% SDS, 50% formamide, and 200 μg/ml ssDNA. Useful variations on these conditions will be readily apparent to those skilled in the art.

The washing steps, which follow hybridization, can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include temperature of at least about 25 degrees C., more preferably of at least about 42 degrees C., and most preferably of at least about 68 degrees C. In a preferred embodiment, wash steps will occur at 25 degree C. in 30 mM NaCl, 3 mM trisodium citrate, and 0.1% SDS. In a more preferred embodiment, wash steps will occur at 42 degrees C. in 15 mM NaCl, 1.5 mM trisodium citrate, and 0.1% SDS. In a most preferred embodiment, wash steps will occur at 68 degrees C. in 15 mM NaCl, 1.5 mM trisodium citrate, and 0.1% SDS. Additional variations on these conditions will be readily apparent to those skilled in the art.

The present invention also encompasses the detection of nucleic acid molecules that hybridize under stringent hybridization conditions (as defined herein) to all or a portion of the nucleotide sequence represented by SEQ ID NO:1 or its complement, wherein the detected nucleic acid molecule encodes a functional PBK protein. The hybridizing portion of the hybridizing nucleic acids is typically at least 15 (e.g., 20, 25, 30, or 50) nucleotides in length. The hybridizing portion of the hybridizing nucleic acid is at least 80%, at least 95%, or at least 98% or more identical to the sequence of a portion or all of a nucleic acid encoding a PBK protein, or its complement. Hybridizing nucleic acids of the type described herein can be used, for example, as a cloning probe, a primer (e.g., a PCR primer), or a diagnostic probe. Additional guidance is readily available in the art, for example, by Sambrook et al., 1989, Molecular Cloning, A Laboratory Manual, Cold Springs Harbor Press, NY; and Ausubel et al. (eds.) 1995, Current Protocols in Molecular Biology, (John Wiley & Sons, NY).

The present invention provides a kit comprising oligonucleotide probes useful in detecting PBK markers, and or antibodies useful in detecting PBK markers, for use in the diagnostic methods provided herein. The oligonucleotide probes are based on the PBK nucleotide sequence and are preferably from about 10, 20, 30, 40, 50 or more nucleotides in length. Such oligonucleotides, and combinations of oligonucleotide pairs, are useful as primers in PCR-based detection of PBK tumor markers and in-situ hybridization of tumor tissue samples. These oligonucleotides may contain one or more modified linking groups, sugar residues and/or base, to increase the oligonucleotide's resistance to degradation. The kit may further contain printed instructions setting forth standard and diagnostic levels of a PBK nucleotide marker in a specific type of tissue. The elements of the test kit are packaged together in any suitable manner, typically with all elements in a single container and including printed instructions for carrying out the test. The test kit may further include a known amount of nucleic acid molecules encoding PBK, to be used as a standard or control.

A further embodiment of the present invention is a test kit comprising antibodies specific for PBK protein, and further containing written instructions setting forth standard and diagnostic levels of PBK proteins in a specific type of tissue. The antibodies may be conjugated to a solid support or conjugated to a detectable element. The elements of the test kit are packaged together in any suitable manner, typically with all elements in a single container and including printed instructions for carrying out the test.

The present invention further provides a method of screening agents for potential pharmaceutical actions, such as anti-cancer effects, where said agent causes a decrease in a PBK marker. Such agents may directly inhibit the expression of the PBK gene at either the transcriptional or translational level, or inhibit PBK activity, or inhibit a step in the biolobical pathway in which PBK operates, resulting in a decrease in a PBK marker. Such methods include exposing cells that overexpress a PBK marker to an agent, and then determining the level of PBK marker in the cells after exposure to the agent. The cells may be in vitro, or in vivo, and may be tumor cells or neoplastic cells. Particularly preferred are tissue culture methods and non-human mammalian models of human cancers. The agent may be administered systemically to the animal or directly to the neoplastic growth. The level of PBK marker is compared to a pre-determined standard or to levels found in a control. A decrease in the level of PBK marker indicates that the agent inhibits PBK gene expression or activity and may have anti-cancer effects. Such screening methods are particularly suitable for use in animal or cell culture models of prostate, breast, lung and colon cancer. Alternatively, such screening methods may be carried out by in vitro screening of a malignant transformed cell line in which the level of a PBK marker is increased compared to non-transformed cells of the same type. The agent may be considered inhibitory if the level of PBK marker is significantly decreased, even though the level of PBK marker continues to be above the standard or normal level. Ideally the level of PBK marker will be decreased by at least 20%, 25% or 50%, and preferably by at least 70% or more, relative to the same cell line which was not exposed to the inhibitory agent. Examples of potential inhibitors of PBK mRNA or protein production include antisense RNA, competitive inhibitors of PBK proteins such as fragments of the PBK protein, or antibodies to the PBK protein. The PBK inhibitor can be directed to a nucleic acid molecule which encodes the PBK protein, i.e., the PBK gene or RNA transcripts thereof, or to the PBK protein itself, or subunits thereof. Substances which can serve as PBK inhibitors include, but are not limited to, compounds that specifically inhibit the interaction between PBK and its substrates, compounds that specifically degrade or inactivate the PBK protein, and compounds that specifically interfere with the expression of PBK protein. Such agents may include chemical compound inhibitors of PBK, protein or peptide PBK antagonists or competitive inhibitors, antibodies to the PBK protein, and molecules that inhibit the expression of PBK such as triplex forming oligonucleotides, antisense oligonucleotides, ribozymes, aptamers, etc.

The present invention also embraces a method of treating cancerous, neoplastic, or hyperproliferative growths in which elevated PBK activity levels occur. The method comprises exposing cancer cells to an agent that inhibits the activity (or expression) of PBK, in an amount sufficient to reduce or inhibit the PBK activity (or expression) that would otherwise be present in the cells (i.e., the activity or expression that would be expected to occur in the absence of the inhibiting agent). Preferably, the PBK inhibiting agent is provided in an amount that reduces PBK activity or expression to a level found in non-diseased cells of the same tissue type. The PBK inhibitor may be administered systemically to a subject suffering from a cancer, neoplasm, or hyperproliferative disorder in which a PBK marker is elevated, or may be administered directly to the growth, e.g., by localized injection. The growth may be, e.g., prostate cancer or breast cancer. The optimal dosage of the PBK inhibitor will vary, depending on factors such as type and extent of progression of the cancer, the overall health status of the patient, the potency of the inhibitor, and route of administration. Optimization of the PBK inhibitor dosage is within the ordinary skill in the art.

The PBK inhibitor can be directed to a nucleic acid molecule which encodes the PBK protein, i.e., the PBK gene or RNA transcripts thereof, or to the PBK protein itself, or subunits thereof. Substances which can serve as PBK inhibitors include, but are not limited to, compounds that specifically inhibit the interaction between PBK and its substrates, compounds that specifically degrade or inactivate the PBK protein, and compounds that specifically interfere with the expression of PBK protein. Such agents may include chemical compound inhibitors of PBK, protein or peptide PBK antagonists, and molecules that inhibit the expression of PBK such as triplex forming oligonucleotides, antisense oligonucleotides, ribozymes, etc.

A mouse PBK kinase cDNA sequence was isolated (SEQ ID NO:7) and the encoded amino acid sequence was determined by the present inventors as:

| | | |
|---|---|---|
| MEGINNFKTP NKSEKRKSVL CSTPCVNIPA SPFMQKLGFG TGVSVYLMKR SPRGLSHSPW | 60 | (SEQ ID NO:8) |
| AVKKISLLCD DHYRTVYQKR LTDEAKILKN LNHPNIIGYR AFTEASDGSL CLAMEYGGEK | 120 | |
| SLNDLIEERN KDSGSPFPAA VILRVALHMA RGLKYLHQEK KLLHGDIKSS NVVIKGDFET | 180 | |
| IKICDVGVSL PLDENMTVTD PEACYIGTEP WKPKEALEEN GIITDKADVF AFGLTLWEMM | 240 | |
| TLCIPHVNLP DDDVDEDATF DESDFDDEAY YAALGTRPSI NMEELDDSYQ KAIELFCVCT | 300 | |
| NEDPKDRPSA AHNVEALELD GQCCGLSSKH | | |

The consensus D-X-K-X-X-N sequence for serine/threonine or dual specificity kinases is underlined, above. GenBank Accession Number AB041882 provides an essentially similar sequence, identified as mouse TOPK (T-cell-originated protein kinase), see also Abe et al., J. Biological Chem. 275(28):21525 (2000). The amino acid sequence provided at GenBank Accession No. AB041882 recites isoleucine (1) at position 313 (indicated by bold underlined type in the above sequence).

Transgenic non-human animals, preferably mice, having germ and/or somatic cells in which at least one allele of an endogenous PBK gene is functionally disrupted are also provided by the present invention. The animal may be heterozygous or, more preferably, homozygous for the PBK gene disruption. In homozygous animals having functional disruption of the endogenous PBK gene, PBK activity is substantially reduced relative to that found in wild-type animals. The transgenic animals of the invention can be used as positive controls to evaluate the efficacy of PBK inhibitors and to identify disease conditions that can be treated with PBK inhibitors. Transgenic non-human animals, preferably mice, having functionally disrupted endogenous PBK genes but which have been reconstituted with a heterologous PBK gene (preferably human) are also provided. Such animals can be used to identify agents that inhibit human PBK activity in vivo. Nucleic acid constructs for functionally disrupting an endogenous PBK gene in a host cell, recombinant vectors including the nucleic acid construct, and host cells into which the nucleic acid construct has been introduced are also encompassed by the invention. Also provided are non-human animals in which expression of endogenous PBK has been increased relative to that seen in wild-type animals; such over-expression may be limited to a particular tissue type or anatomic area of the animal. Also provided are non-human animals in which expression of a heterologous PBK is achieved in a particular tissue type or anatomic area of the animal.

The present invention pertains to nonhuman animals (preferably non-human mammals, and more preferably mice) with somatic and/or germ cells having a functional disruption of at least one, and more preferably both, alleles of an endogenous PBK gene. Accordingly, the invention provides viable animals having an altered PBK gene, and thus lacking PBK activity (or possessing substantially reduced PBK activity compared to that observed in wild-type animals) in at least one tissue type. The non-human animals of the invention are useful, for example, as standard controls by which to evaluate PBK inhibitors, as recipients of a normal human PBK gene to thereby create a model system for screening human PBK inhibitors in vivo, and/or to identify disease states for treatment with PBK inhibitors.

In the transgenic nonhuman animal of the invention, the PBK gene preferably is disrupted by homologous recombination between the endogenous allele and an altered or heterologous PBK gene, or portion thereof, that has been introduced into an embryonic stem cell precursor of the animal. In addition to allowing for introduction of a null mutation into an endogenous PBK gene, similar techniques can be used to introduce point mutations or deletions into a PBK gene allele, to alter PBK gene activity. The embryonic stem cell precursor is then allowed to develop, resulting in an animal having an altered PBK gene, including functionally disrupted PBK genes. The animal may have one PBK gene allele functionally disrupted (i.e., the animal may be heterozygous for this alteration), or more preferably, the animal has both PBK gene alleles functionally disrupted (i.e., the animal can be homozygous for the alteration).

In one embodiment of the invention, functional disruption of both PBK gene alleles produces animals in which expression of the PBK gene product in cells of the animal is substantially absent relative to non-altered animals. In another embodiment, the PBK gene alleles can be disrupted such that an altered (i.e., mutant or non-wild type) PBK gene product is produced in cells of the animal.

Additionally, the animals of the invention are useful for determining whether a particular disease condition involves the action of PBK, and thus can potentially be treated by a PBK inhibitor. For example, an attempt can be made to induce a disease condition in an animal of the invention having a functionally disrupted PBK gene. Subsequently, the susceptibility or resistance of the animal to the disease condition can be determined. A disease condition that is treatable with a PBK inhibitor can be identified based upon resistance of an animal of the invention to the disease condition.

Another aspect of the invention pertains to a transgenic nonhuman animal having a functionally disrupted endogenous PBK gene but which also carries in its genome, and expresses, a transgene encoding a heterologous PBK (i.e., a PBK gene from another species). Preferably, the animal is a mouse and the heterologous PBK is a human PBK, such as a PBK of SEQ ID NO:2 or SEQ ID NO:3 provided herein. An animal of the invention which has been reconstituted with human PBK can be used to identify agents that inhibit human PBK in vivo. For example, the animal can be exposed to a stimulus that induces production of PBK in the presence and absence of an agent to be tested and the PBK response in the animal can be measured. An agent that inhibits human PBK in vivo can be identified based upon a decreased PBK response in the presence of the agent compared to the PBK response in the absence of the agent.

Yet another aspect of the invention pertains to a nucleic acid construct for functionally disrupting a PBK gene in a host cell. The nucleic acid construct comprises: a) a nonhomologous replacement portion; b) a first homology region located upstream of the nonhomologous replacement portion, the first homology region having a nucleotide sequence with substantial identity to a first PBK gene sequence; and c) a second homology region located downstream of the nonhomologous replacement portion, the second homology region having a nucleotide sequence with substantial identity to a second PBK gene sequence, the second PBK gene sequence having a location downstream of the first PBK gene sequence in a naturally occurring endogenous PBK gene. Additionally, the first and second homology regions are of sufficient length for homologous recombination between the nucleic acid construct and an endogenous PBK gene in a host cell when the nucleic acid molecule is introduced into the host cell. A nucleotide sequence with “substantial identity” to a PBK gene sequence is intended to describe a nucleotide sequence having sufficient sequence identity to a PBK gene sequence to allow for homologous recombination between the nucleotide sequence and an endogenous PBK gene sequence in a host cell. Typically, the nucleotide sequences of the flanking homology regions are at least 80%, more preferably at least 90%, even more preferably at least 95% and most preferably 98%, 99% or 100% identical to the nucleotide sequences of the endogenous PBK gene targeted for homologous recombination. Most preferably, the flanking homology regions are isogenic with the targeted endogenous allele (e.g., the DNA of the flanking regions is isolated from cells of the same genetic background as the cell into which the targeting construct is to be introduced).

In a preferred embodiment, the nonhomologous replacement portion comprises a positive selection expression cassette, as is known in the art, preferably including a neomycin phosphotransferase gene operatively linked to a regulatory element(s). The term “positive selection expression cassette” refers to nucleotide sequences encoding a positive selection marker operatively linked to regulatory elements that control expression of the positive selection marker (e.g., promoter and polyadenylation sequences). A “positive selection marker” allows for selection of cells which contain the marker. In another preferred embodiment, the nucleic acid construct also includes a negative selection expression cassette distal to either the upstream or downstream homology regions. A preferred negative selection cassette includes a herpes simplex virus thymidine kinase gene operatively linked to a regulatory element(s).

Another aspect of the invention pertains to recombinant vectors into which the nucleic acid construct of the invention has been incorporated. Yet another aspect of the invention pertains to host cells into which the nucleic acid construct of the invention has been introduced to thereby allow homologous recombination between the nucleic acid construct and an endogenous PBK gene of the host cell, resulting in functional disruption of the endogenous PBK gene. The host cell can be a mammalian cell that normally expresses PBK, or a pluripotent cell, such as a mouse embryonic stem cell. Further development of an embryonic stem cell into which the nucleic acid construct has been introduced and homologously recombined with the endogenous PBK gene produces a transgenic nonhuman animal having cells that are descendant from the embryonic stem cell and thus carry the PBK gene disruption in their genome. Animals that carry the PBK gene disruption in their germline can then be selected and bred to produce animals having the PBK gene disruption in all somatic and germ cells. Such mice can then be bred to homozygosity for the PBK gene disruption.

As used herein, a gene that is “functionally disrupted” has a sequence alteration that prevents the normal function of the gene, e.g., prevents expression of a normal PBK gene product or prevents expression of normal amounts of the PBK gene product. The mutation causing the functional disruption can be an insertion, deletion or point mutation(s). In one embodiment, both PBK gene alleles are functionally disrupted such that expression of the PBK gene product is substantially reduced or absent in cells of the animal The term “absent” is intended to mean that essentially undetectable amounts of normal PBK gene product are produced in cells of the animal. This type of mutation is also referred to in the art as a “null mutation” and an animal carrying such a null mutation is also referred to as a “knockout animal”. In another embodiment, both PBK gene alleles are functionally disrupted such that an altered form of the PBK gene product is expressed in cells of the animal. For example, one or more point mutations or deletion mutations can be introduced into the PBK gene to thereby alter the amino acid sequence of the PBK gene product encoded therein. Further, by linking a PBK-encoding DNA sequence to a selected tissue-specific promoter and/or enhancer and introducing by standard methods the resultant hybrid DNA molecule into an animal embryo at an early developmental stage (e.g., the fertilized oocyte stage), a transgenic animal which expresses elevated levels of PBK in the selected tissue (e.g., breast, prostate) can be produced.

It will be apparent to those skilled in the art that a nonhomologous replacement portion can be inserted at various locations within the PBK gene, and flanked by different homology regions, to thereby functionally disrupt the gene. The functional disruption of the mPBK gene sequence may prevent expression of a full-length mPBK mRNA transcript (e.g. by insertion of the neo gene) or may lead to expression of an mPBK mRNA transcript that encodes an altered form of mPBK.

Non-human animals with reduced levels of PBK expression may further be obtained using recombinase technology, as is known in the art, in which a recombinase targeted to the endogenous PBK sequence excises or rearranges the DNA sequence. See e.g., U.S. Pat. No. 6,120,764 regarding adenovirus vector systems that express a recombinase in order to regulate expression of a targetted gene.

Additionally, using viral gene vectors as are known in the art, non-human animals (preferably mice) overexpressing endogenous PBK, or expressing heterologous PBK, may be provided. Preferably, the heterologous PBK is human PBK. Expression of a heterologous PBK, or over-expression of the endogenous PBK, may be limited to a particular tissue type or area of tissue in the animal. Various promoter/enhancers that drive tissue-specific gene expression, and that are useful in providing transgenic non-human mammals, have been identified. An expression cassette contains the regulatory elements necessary for expression of an inserted gene, including a promoter and polyadenylation site and, if necessary, an enhancer element.

Promoter elements within the 5′ DNA region of the rat C(3)1 may be used to direct prostate-specific expression of gene products, and to produce transgenic mice. See, e.g., Zhang et al., Prostate 2000 Jun. 1;43(4):278-85. The promoter and 5′ region of the rat C(3)1 gene is fused to the coding region of the PBK gene using recombinant DNA techniques. Prostates from males can be examined for expression of recombinant PBK mRNA by in situ hybridization

The human prostate-specific antigen (PSA) and glandular kallikrein-1 (hGK-1, also known as hK2) genes are tandemly located on chromosome 19, separated by a 12-kb intergenic region. Transgenes placed under the control of either the PSA gene regulatory elements alone, or together with the intergenic region, exhibit prostate-specific expression. The intergenic region reduces the need for high PSA gene copy-number for high levels of expression. See, e.g., See, e.g., Cleutjens et al., Mol. Endocrinol. 11(9):1256-65 (1997); Wei et al., Int. J. Mol. Med. 2(4):487-496 (1998).

Prostate-specific expression of targeted genes in transgenic mice can also be achieved using fragments of the probasin (PB) promoter. A small PB fragment is sufficient to direct prostate-specific expression, however, use of a larger fragment increases the levels of transgene expression. See e.g., Yan et al., Prostate 32:129-139 (1997).

Thus, the present invention further provides mouse models of tissue-specific overexpression of PBK, using tissue-specific promoters as are known in the art. A preferred tissue-specific promoter is a prostate-specific promoter. Such mice may, for example, express human PBK in a particular tissue such as prostate, or may over-express murine PBK in a particular tissue. These animals are useful in screening compounds for their effects on PBK activity.

Various systems are known in the art to produce transgenic non-human mammals in which the transgene is inducibly regulated. Regulation of PBK expression in the transgenic animals of the present invention may be accomplished using components of the Tet (tetracycline) repressor/operator/inducer system of prokaryotes, as is known in the art. See e.g., U.S. Pat. No. 5,965,440; U.S. Pat. No. 5,922,927; U.S. Pat. No. 5,917,122; U.S. Pat. No. 5,851,796; U.S. Pat. No. 5,650,298.

Alternatively, expression of the PBK gene in a non-human animal can be reduced or ablated by insertion of a ‘gene switch’ such as that described in Bond et al., Science 289:1942-46 (2000). Bond et al. used homologous recombination to insert a doxycycline based gene switch to regulate expression of the targeted gene. Expression of the targeted gene is abolished when the animals are fed doxycycline. The regulatory cassette is inserted into the 5′ untranslated region of the targeted gene.

Additionally, human PBK gene function may be altered in human cells in vitro or in non-human animals containing human tissue implants. Such altered human cells are useful in screening compounds for pharmaceutical effects on PBK activity. To target a human PBK (hPBK) gene in a human host cell by homologous recombination, the targeting vector includes flanking homology regions having substantial identity to human PBK gene sequences. Human PBK genomic DNA sequences can be isolated by screening a human genomic DNA library with a cDNA probe encompassing all or part of the human PBK cDNA using standard techniques. The nucleotide sequence of a human PBK cDNA and predicted amino acid sequence of a human PBK protein are provided by SEQ ID NOs: 1 and 2, respectively. As described for the mouse PBK gene, the functional disruption of the human PBK gene sequence in a human cell may prevent expression of a full-length hPBK mRNA transcript or may lead to expression of an hPBK mRNA transcript that encodes an altered form of hPBK.

To functionally disrupt an endogenous PBK gene allele in a host cell, a targeting vector of the invention is introduced into the host cell, e.g., a differentiated cell that normally expresses PBK or an embryonic stem cell, and homologous recombinants are selected. A targeting vector can be introduced into a host cell by any of several techniques known in the art suitable for the introduction of exogenous DNA (e.g., calcium phosphate precipitation, DEAE-dextran transfection, microinjection, lipofection and the like) but is most preferably introduced into the host cell by electroporation. After introduction of the vector into the host cell, the cell is cultured for a period of time and under conditions sufficient to allow for homologous recombination between the introduced targeting vector and an endogenous PBK gene. Host cells are selected and screened for homologous recombination at the endogenous PBK gene locus by standard techniques (e.g., Southern hybridizations using a probe which distinguishes the normal endogenous allele from the homologous recombinant allele).

### EXAMPLES

### Quantification of mRNA

A quantitative assay to detect PBK mRNA and compare the relative amounts of PBK mRNA in tumor tissue and normal (non-tumorous) tissue was designed. The assay used real-time fluorescent-probe polymerase chain reaction (PCR) (TaqMan).

The TaqMan sequence detector (ABI PRISM 7700 Sequence Detector) integrates a PCR-based assay and hardware/software instrumentation to provide high-throughput quantitation of nucleic acid sequences. The combination of thermal cycling, fluorescence detection, and application-specific software allows the cycle-by-cycle detection of the increase in the PCR product. Quantitative results are provided.

The method used a fluorogenic probe complementary to the target sequence, which was added to the PCR reaction mixture. The probe comprised an oligonucleotide with a reporter and quencher dye attached. If the target nucleic acid was present during PCR, the probe annealled specifically between the forward and reverse primer sites. The polymerase cleaved the probe, releasing the reporter dye from the influence of the quencher dye, and thus causing an increase in the fluorescent intensity of the reporter dye. Fluorescent emission was then recorded and/or measured. See, e.g., Orlando, C., P., Pinzani, and M., Pazzagli. “Developments in quantitative PCR.” Clin Chem Lab Med 36 (1998): 255-269; Brink et al., “Comparative quantification of IL-1beta, IL-10, IL-10r, TNFalpha and IL-7 mRNA levels in UV-irradiated human skin in vivo”, Inflamm Res 49(6):290 (2000).

Primer & Probe Sequences:

| | | |
|---|---|---|
| Forward Primer: | 5′ AGT TAA CAG TTC CCA TAC AAG TTA ATG C 3′ | (SEQ ID NO:4) |
| Reverse Primer: | 5′ CAC ACA TCG TTG AAG CTT TGG A 3′ | (SEQ ID NO:5) |
| Probe: | 5′-(FAM)-TTG AGC TTA GAC CAC AAC ATT GGC CAT CTA G-(TAMRA)-3′ | (SEQ ID NO:6) |

FAM refers to the dye used; TAMRA refers to the quencher.

PCR Cycling Conditions:

48° C. 30 minutes
95° C. 10 minutes
40 cycles of:
94° C. 15 seconds
60° C. 1 minute

The specific reagents and conditions used for Taqman analyses are shown in Table 1.

TABLE 1

| | Final | Volume (μl) | Number of |
|---|---|---|---|
| | Concentration | per Rxn | Reactions = 96 |
| 25 mM MgCl2 | 5.5 mM | 11 | 1056 |
| 10× Taqman Buffer A | 1× | 5 | 480 |
| DNTP Mix (10 mM) | 300 μM | 1.5 | 144 |
| Rnase inhibitor (20 U/μl) | | 1 | 96 |
| MuLV R'tase (50 U/μl) | | 0.25 | 24 |
| 3 μM Forward Primer | 300 nM | 5 | 480 |
| 3 μM Reverse Primer | 300 nM | 5 | 480 |
| Amplitaq Gold (5 U/μl) | | 0.25 | 24 |
| Taqman Probe (1.5 mM) | 150 nM | 5 | 480 |
| Template | 50 ng | 5 | |
| | total RNA | | |
| dH20 | | 11 | 1056 |
| | | 50 μl | 4320 |

45 μl of master mix and 5 μl of template

Briefly, a “master mix” of reagents, lacking only the RNA derived from the tissue samples, was created. An aliquot of 45 μL of master mix was added to each well of a 96 well Taqman plate. The specific sample RNA was then added to the appropriate wells (50 ng RNA in 5 μL total volume of water), each sample in triplicate. The Taqman plate was covered per manufacturers instructions and placed within the ABI PRISM 7700 Sequence Detector. Analysis was performed with the primers and probe and under the PCR conditions described above.

### Samples

Samples of normal human tissue were obtained (surgical samples from local hospitals, except brain and fetal brain which were autopsy samples) for Adenoid, Adipose, Adrenal, Brain, Breast, Cervix, Colon, Endometrium, Heart, Hypothalamus, Ileum, Jejunum, Kidney, Liver, Lung, Myometrium, Ovary, Pancreas, Head of Pancreas, Placenta, Prostate, Rectum, Skeletal Muscle, Skin, Small Intestine, Spleen, Stomach, Testis, Thyroid, Tonsil, Urinary Bladder, Uterus, Fetal Brain, and Fetal Liver.

Paired samples of histologically diagnosed tumor tissue and normal tissue were obtained. As used herein, ‘paired’ samples are samples of tumor tissue and normal tissue obtained from the same subject. Twenty-three samples of lung tumor; nine samples of prostate tumors; twenty samples of colon tumors; and nine samples of breast tumors were studied.

### Results: Normal Tissue

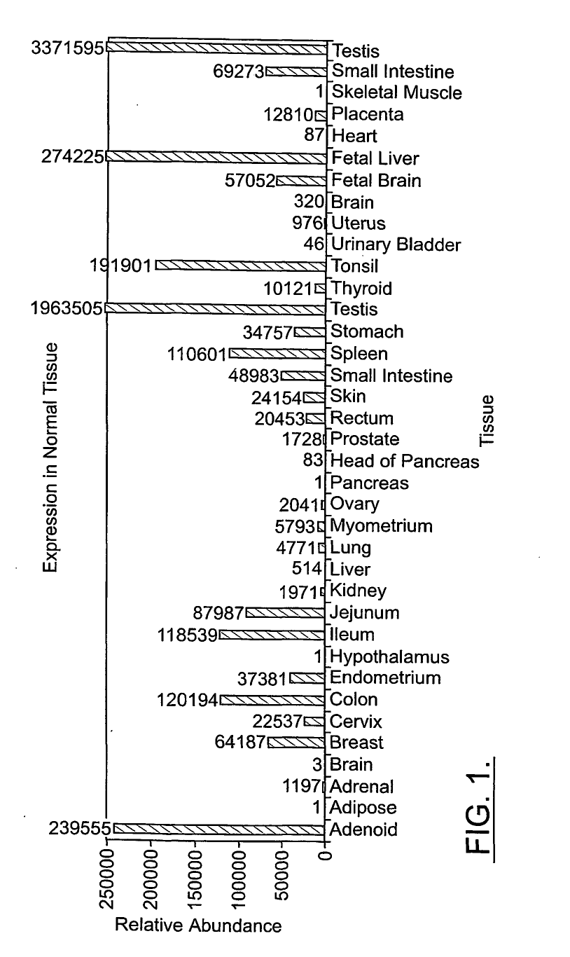

The relative abundance of PBK mRNA in various normal (non-cancerous) tissue types was determined using fluorescent-probe polymerase chain reaction as described above. Results are shown in FIG. 1. PBK mRNA was most abundant in testis.

### Results: Lung Tumor Tissue

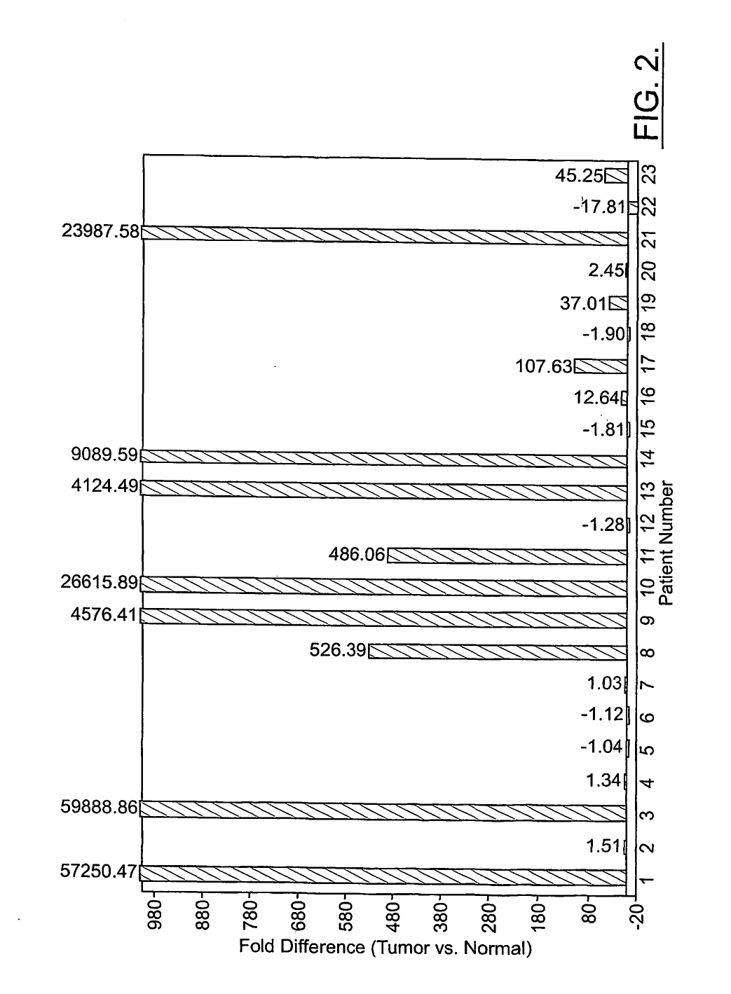

PBK mRNA in lung tumor samples was compared to that in paired normal lung tissue samples. As shown on FIG. 2, in 13 of 23 (57%) lung tumor samples, PBK mRNA was expressed at least ten-fold higher when compared to its paired normal lung tissue.

### Results: Prostate Tumor Tissue

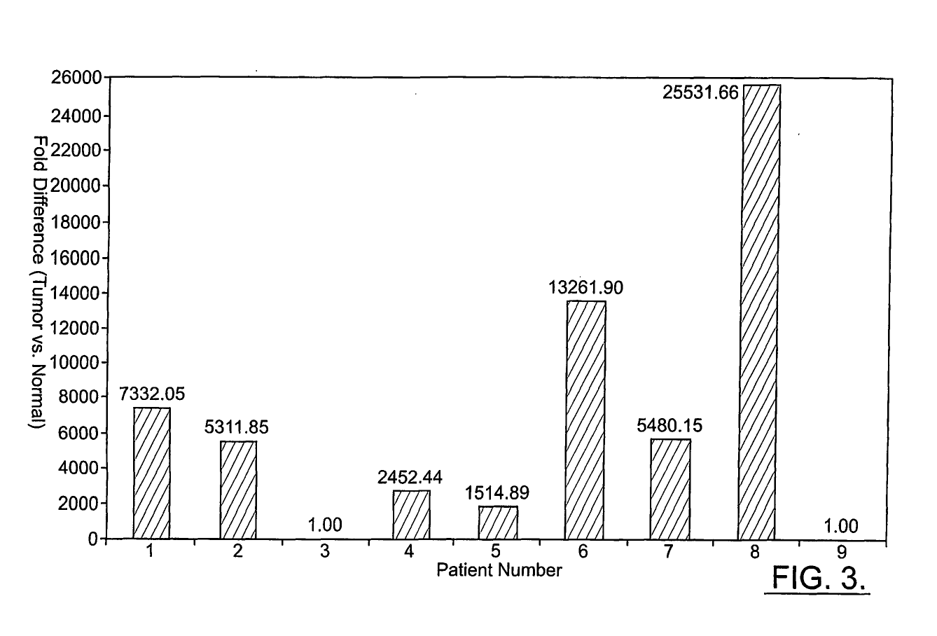

PBK mRNA in prostate tumor samples was compared to that in paired normal prostate tissue samples. As shown on FIG. 3, in 7 of 9 (78%) prostate tumor samples, PBK mRNA was expressed at least ten-fold higher when compared to its paired normal prostate tissue. In the other two tumor samples, PBK mRNA levels in the tumor samples were the same (“0”) as that detected in normal tissue.

### Results: Colon Tumor Tissue

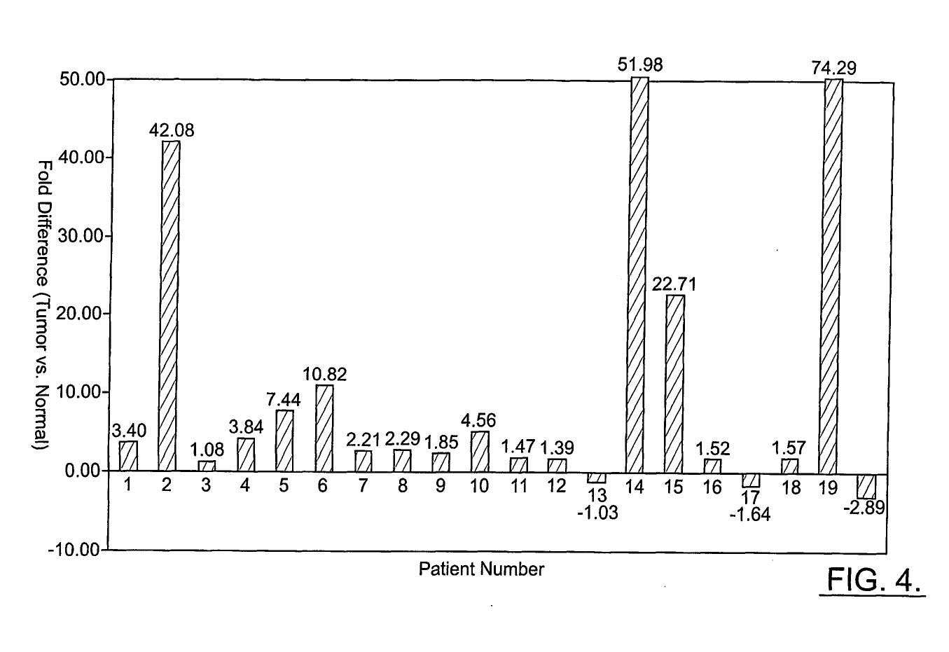

PBK mRNA in colon tumor samples was compared to that in paired normal colon tissue samples. As shown on FIG. 4, in 5 of 20 (25%) lung tumor samples, PBK mRNA was expressed at least ten-fold higher when compared to its paired normal colon tissue.

### Results: Breast Tumor Tissue

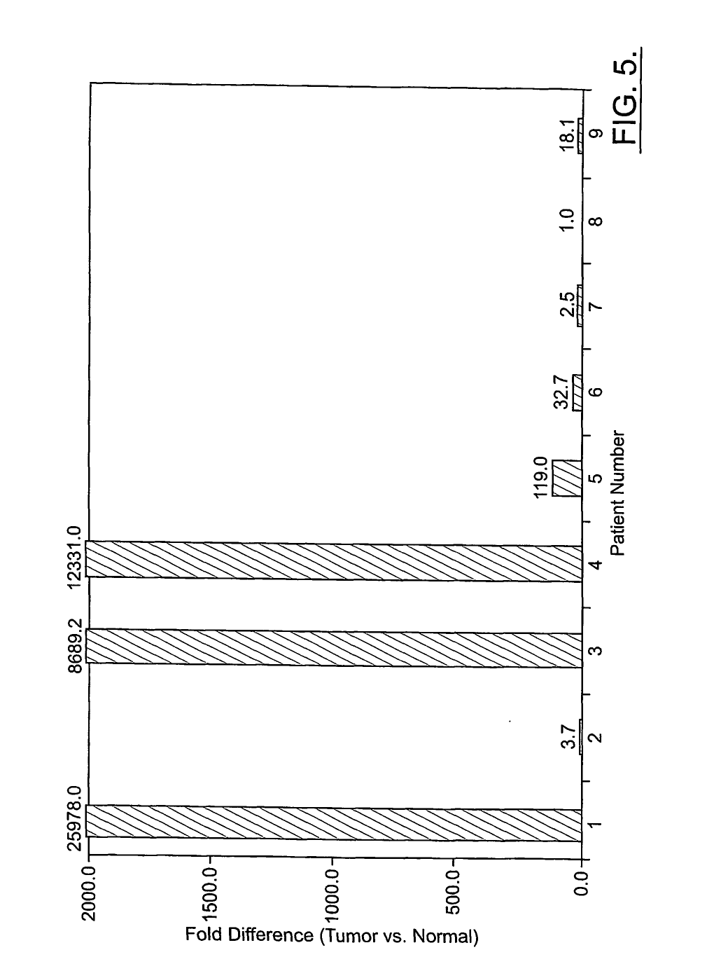

PBK mRNA in breast tumor samples was compared to that in paired normal breast tissue samples. As shown on FIG. 5, in 6 of 9 (67%) breast tumor samples, PBK mRNA was expressed at least ten-fold higher when compared to its paired normal breast tissue.

1. A method of detecting mRNA encoding a marker protein associated with a cancer that is characterized by over-expression of PDZ-binding kinase (PBK) compared to non-cancerous cells, comprising:
(a) obtaining a test sample of tissue suspected of being cancerous;
(b) contacting mRNA from cells in said test sample tissue with at least one nucleotide probe that is complementary to and specifically hybridizes to mRNA encoding PBK, to form a hybridization product;
(c) detecting the amount of hybridization product produced; and
(d) comparing the amount of hybridization product detected in (c) to the amount expected in non-malignant cells;
where a finding that the amount of hybridization product produced in (c) is greater than that produced by non-malignant control cells indicates the presence of cancerous cells in said test sample.

2. A method according to claim 1 where said tissue is selected from prostate, breast, lung and colon.

3. A method according to claim 1 where said tissue is selected from prostate and breast tissues.

4. A method according to claim 1 wherein said nucleotide probe comprises at least 20 contiguous nucleotides of SEQ ID NO:1.

5. A method according to claim 1 wherein said mRNA encoding PBK encodes a protein of SEQ ID NO:2 or SEQ ID NO:3.

6. A method according to claim 1 where said test sample is obtained from a human subject.

7. A method according to claim 1 where the amount of hybridization product that is detected is compared to an amount detected in a sample of non-malignant tissue obtained from the same subject.

8. A method according to claim 1 where the amount of hybridization product that is detected is compared to a predetermined standard amount.

9. A method of detecting mRNA encoding a marker protein associated with a cancer that is characterized by over-expression of PDZ-binding kinase (PBK) compared to non-cancerous cells, comprising:
(a) obtaining a test sample of tissue suspected of being cancerous;
(b) amplifying an mRNA sequence from cells in said test sample using a nucleic acid sequence primer that is complementary to and specifically hybridizes to mRNA encoding PBK to form an amplified product;
(c) detecting the amount of amplified product produced; and
(d) comparing the amount of amplified product detected in (c) to the amount expected in non-malignant cells;
where a finding that the amount of amplified product produced in (c) is greater than that produced by non-malignant control cells indicates the presence of cancerous cells in said test sample.

10. A method according to claim 9 where said tissue is selected from prostate, breast, lung and colon.

11. A method according to claim 9 where said tissue is selected from prostate and breast tissues.

12. A method according to claim 9 wherein said nucleotide probe comprises at least 20 contiguous nucleotides of SEQ ID NO:1.

13. A method according to claim 9 wherein said mRNA encoding PBK encodes a protein of SEQ ID NO:2 or SEQ ID NO:3.

14. A method according to claim 9 where said test sample is obtained from a human subject.

15. A method according to claim 9 where the amount of hybridization product that is detected is compared to an amount detected in a sample of non-malignant tissue obtained from the same subject.

16. A method according to claim 9 where the amount of hybridization product that is detected is compared to a predetermined standard amount.

17. A method according to claim 9, where the amplification is by RT-PCR.

18. A method of screening a sample of prostate tissue for malignancy, comprising measuring the amount of PBK mRNA in said sample tissue, wherein an elevated level of PBK mRNA in said sample, compared to non-malignant prostate tissue, indicates that said sample tissue is malignant.

19. A method of screening a sample of prostate tissue for malignancy, comprising measuring the amount of PBK protein in said sample tissue, wherein an elevated level of PBK in said sample, compared to non-malignant prostate tissue, indicates that said sample tissue is malignant.

20. A method of screening a sample of breast tissue for malignancy, comprising measuring the amount of PBK mRNA in said sample tissue, wherein an elevated level of PBK mRNA in said sample, compared to non-malignant breast tissue, indicates that said sample tissue is malignant.

21. A method of screening a sample of breast tissue for malignancy, comprising measuring the amount of PBK protein in said sample tissue, wherein an elevated level of PBK in said sample, compared to non-malignant breast tissue, indicates that said sample tissue is malignant.

22. A method of screening a test compound for the ability to inhibit activity of PBK in cells, comprising:
(a) exposing a cell expressing PBK to a test compound;
(b) measuring the amount of PBK activity in said cells; and
(c) comparing the amount of PBK activity in said exposed test cells to that in control cells that are not exposed to said test compound;
wherein a decreased amount of PBK activity in said exposed test cells compared to control cells indicates that said test compound inhibits PBK activity in cells.

23. A method according to claim 22, wherein said exposing step is carried out ill vitro.

24. A method according to claim 22, wherein said exposing step is carried out in vivo.

25. A kit for detecting mRNA encoding a marker protein associated with a human cancer characterized by over-expression of PDZ-binding kinase (PBK) compared to non-cancerous cells, the kit comprising:
(a) a labeled nucleic acid molecule at least 20 nucleotides in length and complementary to a human PBK mRNA sequence, said nucleic acid molecule able to specifically hybridize to said human PBK mRNA sequence to form a labeled hybridization product; and
(b) printed instructions setting forth levels of PBK mRNA expected in cancerous cells of at least one human tissue type.

26. A kit according to claim 25, further comprising a known amount of mRNA encoding human PBK, for use as a control or standard.

27. A kit according to claim 25 wherein said printed instructions set forth comparative levels of PBK mRNA found in non-cancerous and cancerous prostate tissue cells.

28. A kit according to claim 25 wherein said printed instructions set forth comparative levels of PBK mRNA found in non-cancerous and cancerous breast tissue cells.

29. A kit according to claim 25 where said probe is complementary to a portion of SEQ ID NO:1.

30. A kit for detecting a marker protein associated with a human cancer characterized by over-expression of PDZ-binding kinase (PBK) compared to expression levels in non-cancerous cells, the kit comprising:
(a) a labeled antibody that specifically binds to human PBK; and
(b) printed instructions setting forth levels of PBK expected in cancerous cells of at least one human tissue type.

31. A kit according to claim 30 comprising a labeled monoclonal antibody.

32. A kit according to claim 30 comprising a labeled polyclonal antibody

33. A kit according to claim 30 wherein said human tissue type is prostate tissue.

34. A kit according to claim 30 wherein said human tissue type is breast tissue.

35. A kit according to claim 30 wherein said labeled antibody comprises a label selected from the group consisting of enzyme labels, radiolabels and fluorescent labels.

36. A transgenic mouse whose genome comprises a disruption in at least one allele of its endogenous PDZ-binding kinase (PBK) gene, wherein said disruption prevents the expression of a fully functional PBK protein, and wherein said disruption results in said transgenic mouse exhibiting decreased levels of PBK activity as compared to a wild-type mouse.

37. The transgenic mouse of claim 36, wherein the disruption results from insertion of a positive selection expression cassette into the endogenous P13K gene.

38. A method for producing a transgenic mouse exhibiting decreased levels of PBK activity relative to a wild-type mouse, said method comprising:
(a) introducing a PBK targeting vector into a mouse embryonic stem cell;
(b) introducing said mouse embryonic stem cell into a mouse blastocyst;
(c) transplanting said mouse blastocyst into a pseudopregnant mouse;
(d) allowing said transplanted mouse blastocyst to develop to term;
(e) identifying a transgenic mouse whose genome comprises a disruption of the endogenous PBK gene in at least one allele.

39. A method according to claim 38, further comprising breeding the transgenic mouse of step (e) to obtain a transgenic mouse whose genome comprises a disruption of both alleles of the endogenous PBK gene, wherein said disruption results in said transgenic mouse exhibiting decreased levels of PBK activity relative to a wild-type mouse.

40. The method of claim 39, wherein said PBK targeting vector comprises a positive selection expression cassette.

41. A transgenic mouse whose genome comprises a transgene comprising a DNA sequence encoding human PBK operably linked to a tissue-specific promoter, wherein said DNA sequence is expressed in at least some cells of said tissue.

42. A transgenic mouse according to claim 41 where said develops neoplasia or hyperplasia in said tissue.

43. The transgenic mouse of claim 41, wherein said tissue specific promoter is a prostate tissue specific promoter.


SEQUENCE LISTING (8 sequences)

SEQ ID NO: 1 - DNA, 1804 bp, Homo sapiens
  CDS (137)..(1105)
ggtgctagaggcagctgcagggtctcgctgggggccgctcgggaccaattttgaagaggtacttggccacgacttattttcacctccgacctttccttccaggcggtgagactctggactgagagtggctttcacaatggaagggatcagtaatttcaagacaccaagcaaattatcagaaaaaaagaaatctgtattatgttcaactccaactataaatatcccggcctctccgtttatgcagaagcttggctttggtactggggtaaatgtgtacctaatgaaaagatctccaagaggtttgtctcattctccttgggctgtaaaaaagattaatcctatatgtaatgatcattatcgaagtgtgtatcaaaagagactaatggatgaagctaagattttgaaaagccttcatcatccaaacattgttggttatcgtgcttttactgaagccaatgatggcagtctgtgtcttgctatggaatatggaggtgaaaagtctctaaatgacttaatagaagaacgatataaagccagccaagatccttttccagcagccataattttaaaagttgctttgaatatggcaagagggttaaagtatctgcaccaagaaaagaaactgcttcatggagacataaagtcttcaaatgttgtaattaaaggcgattttgaaacaattaaaatctgtgatgtaggagtctctctaccactggatgaaaatatgactgtgactgaccctgaggcttgttacattggcacagagccatggaaacccaaagaagctgtggaggagaatggtgttattactgacaaggcagacatatttgcctttggccttactttgtgggaaatgatgactttatcgattccacacattaatctttcaaatgatgatgatgatgaagataaaacttttgatgaaagtgattttgatgatgaagcatactatgcagccttgggaactaggccacctattaatatggaagaactggatgaatcataccagaaagtaattgaactcttctctgtatgcactaatgaagaccctaaagatcgtccttctgctgcacacattgttgaagctctggaaacagatgtctagtgatcatctcagctgaagtgtggcttgcataaataactgtttattccaaaatatttacatagttactatcagtagttattagactctaaaattggcatatttgaggaccatagtttcttgttaacatatggataactatttctaatatgaaatatgcttatattggctataagcacttggaattgtactgggttttctgtaaagttttagaaactagctacataagtactttgatactgctcatgctgacttaaaacactagcagtaaaacgctgtaaactgtaacattaaattgaatgaccattacttttattaatgatctttcttaaatattctatattttaatggatctactgacattagcactttgtacagtacaaaataaagtctacatttgtttaaaacactgaaccttttgctgatgtgtttatcaaatgataactggaagctgaggagaatatgcctcaaaaagagtagctccttggatacttcagactctggttacagattgtcttgatctcttggatctcctcagatctttggtttttgctttaatttattaaatgtattttccatactgagtttaaaatttattaatttgtaccttaagcatttcccagctgtgtaaaaacaataaaactcaaataggatgataaagaataaaggacactttgggtaccaaaaaaaaaaaaaaaaaaa

SEQ ID NO: 2 - protein, 322 aa, Homo sapiens
MEGISNFKTPSKLSEKKKSVLCSTPTINIPASPFMQKLGFGTGVNVYLMKRSPRGLSHSPWAVKKINPICNDHYRSVYQKRLMDEAKILKSLHHPNIVGYRAFTEANDGSLCLAMEYGGEKSLNDLIEERYKASQDPFPAAIILKVALNMARGLKYLHQEKKLLHGDIKSSNVVIKGDFETIKICDVGVSLPLDENMTVTDPEACYIGTEPWKPKEAVEENGVITDKADIFAFGLTLWEMMTLSIPHINLSNDDDDEDKTFDESDFDDEAYYAALGTRPPINMEELDESYQKVIELFSVCTNEDPKDRPSAAHIVEALETDV

SEQ ID NO: 3 - protein, 322 aa, Homo sapiens
MEGISNFKTPSKLSEKKKSVLCSTPTINIPASPFMQKLGFGTGVNVYLMKRSPRGLSHSPWAVKKINPICNDHYRSVYQKRLMDEAKILKSLHHPNIVGYRAFTEASDGSLCLAMEYGGEKSLNDLIEERYKASQDPFPAAIILKVALNMARGLKYLHQEKKLLHGDIKSSNVVIKGDFETIKICDVGVSLPLDENMTVTDPEACYIGTEPWKPKEAVEENGVITDKADIFAFGLTLWEMMTLSIPHINLSNDDDDEDLTFDESDFDDEAYYAALGTRPPINMEELDESYQLVIELFSVCTNEDPKDRPSAAHIVEALETDV

SEQ ID NO: 4 - DNA, 28 bp, Artificial Sequence
  Forward primer for detection of human PBK mRNA
agttaacagttcccatacaagttaatgc

SEQ ID NO: 5 - DNA, 22 bp, Artificial Sequence
  Reverse primer for detection of human PBK mRNA
cacacatcgttgaagctttgga

SEQ ID NO: 6 - DNA, 31 bp, Artificial Sequence
  Probe for detection of human PBK mRNA
ttgagcttagaccacaacattggccatctag

SEQ ID NO: 7 - DNA, 1739 bp, Mus musculus
  CDS (263)..(1255)
tgcgagtgttggcgggagggagctgttcctgcattttctggagcgagtcttctgactgcttttagttagaactccagtgcccctcggcgggccgcggcctttgaaaatgcgcgcgccctaaacgctgcggcggttacgctgttagcgggagggagctgagcctgcactttccggactaggtgtccagacagctttgagccagcccagtcactttcacctttttacccgagcgtgcgagcgtggacctaacgtgattgctacaatggaaggaattaataatttcaagacgccaaacaaatctgaaaaaaggaaatctgtattatgttccactccatgtgtaaatatccctgcctctccatttatgcagaagcttggctttgggactggggtcagcgtttacctaatgaaaagatctccaagagggttgtctcattctccttgggccgtgaaaaagataagtcttttatgcgatgatcattatcgaactgtgtatcagaagagactaactgatgaagctaagattttaaaaaaccttaatcacccaaacattataggatatcgtgcttttactgaagccagtgatggtagtctgtgccttgctatggagtatggaggtgaaaagtctctgaatgacttaatagaagagcggaacaaagacagtggaagtccttttccagcagctgtaattctcagagttgctttgcacatggccagagggctaaagtacctgcaccaagaaaagaagctgcttcatggagacataaagtcttcaaatgttgtaattaaaggtgattttgaaacaattaaaatctgtgatgtaggagtctctctgccattggatgaaaatatgactgtgactgatcctgaggcctgttatattggtactgagccatggaaacccaaggaagcgttggaagaaaatggcatcattactgacaaggcagatgtgtttgcttttggccttactctgtgggaaatgatgactttatgtattccacacgtcaatcttccagatgatgatgttgatgaagatgcaacctttgatgagagtgacttcgatgatgaagcatattatgcagctctggggacaaggccatccatcaacatggaagagctggatgactcctaccagaaggccattgaactcttctgtgtgtgcactaatgaggatcctaaagatcgcccgtctgctgcacacaacgttgaagctttggaactagatggccaatgttgtggtctaagctcaaagcattaacttgtatgggaacttgttaactagatatatgtagttaatataacttatggtagctagattctagaagtagctttaacactagtgacccctgtctaagatgacttaagaatcaagggaccattgctttgttacagatctttttagatattcttgcttctttagtgggttactaaaaatttcactacgtacatgtggtacagatatctgtctgctcatagtgtcagtccttcagctggcctgtcaggcccatgcgccctgggacttgagaagagttcataaacggtagctcctagggtgtcttgcctctctacacttagcttctaatttattactttgtttctactgattgtgccttaagtcttttaaaataaatgtaagaataaacaataaaagacagttttagtaccaggaaaaaaaaaaaaaaaaaaaaggccacatgtgctccagctgcaggtcgcggccgctagactagtctagagaaaaa

SEQ ID NO: 8 - protein, 330 aa, Mus musculus
MEGINNFKTPNKSEKRKSVLCSTPCVNIPASPFMQKLGFGTGVSVYLMKRSPRGLSHSPWAVKKISLLCDDHYRTVYQKRLTDEAKILKNLNHPNIIGYRAFTEASDGSLCLAMEYGGEKSLNDLIEERNKDSGSPFPAAVILRVALHMARGLKYLHQEKKLLHGDIKSSNVVIKGDFETIKICDVGVSLPLDENMTVTDPEACYIGTEPWKPKEALEENGIITDKADVFAFGLTLWEMMTLCIPHVNLPDDDVDEDATFDESDFDDEAYYAALGTRPSINMEELDDSYQKAIELFCVCTNEDPKDRPSAAHNVEALELDGQCCGLSSKH